(19) United States
(12) Patent Application Publication
(10) Pub. No.: US 2008/0249502 A1
(43) Pub. Date: Oct. 9, 2008

(54) Apparatus and method of safe placement of bulking materials for gastroscopic vagus nerve bulking to reduce appetite and to effect weight loss

(76) Inventors: Ronald J. Thompson, Fort Thomas, KY (US); James M. Thompson, Cincinnati, OH (US)

Correspondence Address: Donald N. Halgren, 35 Central Street, Manchester, MA, 01944, US

(21) Appl. No.: 12/079,132

(22) Filed: Mar. 25, 2008

Related U.S. Application Data
(63) Continuation-in-part of application No. 11/165,075, filed on Jun. 22, 2005.

(51) Int. Cl. A61M 5/42

(52) U.S. Cl. 604/506; 604/115

(57) ABSTRACT

An apparatus for grasping the fundal mucosa and method for delivery of and safe placement of a tissue bulking agent adjacent to the right and left vagus nerve trunk in the lesser curvature of the of the stomach or at the gastroesophageal junction to compress the vagus nerve trunks that control both electrical stimulation of the gastric ghrelin cells, and the parastolic muscular tone of the smooth muscles of the stomach to effect appetite and effect weight loss.

This application is a continuation-in-part application of co-pending U.S. patent application Ser. No. 11/165,075, filed Jun. 22, 2005, which is incorporated herein by reference in its entirety.

### BACKGROUND OF THE INVENTION

1. Field of the Invention

This invention relates to methods of reducing appetite to effect weight loss.

2. Prior Art

U.S. Pat. Nos. 4,857,057, 6,083,202 and 6,203,533 teach of endoscopic injection systems that prevent over penetration of the sharp distal needle tip by incorporating a shouldered stop at a specific location proximal to the distal needle tip. Almost exclusively, these endoscopic injection systems are utilized to stop active bleeding from gastric or esophageal varicies or from gastric or esophageal biopsy sites. Sclerosing agents or vasoconstrictive agents are directly injected into the actively bleeding vessels to obtain hemostasis. In both situations, either a bleeding varicocele or biopsy site, the bleeding is just below the gastric or esophageal mucosa. The rationale of the safety needle is to prevent over penetration of the sharp needle tip that could insite bleeding from the blood vessels beneath or adjacent to the vessel to be sclerosed or constricted.

The U.S. Pat. No. 7,201,757 describes the endoscopic injection of a bulking agent in multiple injections at the gastro-esophageal junction (GEJ) to prevent gastro esophageal reflux disease (GERD), commonly referred to as heartburn.

U.S. Pat. Nos. 5,716,392; 6,146,391; 6,477,423; (U.S. Pat. No. 6,608,518** Not Right Number); 6,510,332; 6,542,776; and 6,606,523 describe laparoscopic methodology and define and describe the rationale and electronic methodology to present an electrical current to the vagal pacemaker of the fundus of the stomach to reduce appetite and therefore reduce weight. The electrode is a single coated element that presents both negative and positive electrodes in a linear fashion separated by insulated material. This electrically modulated vagus nerve activity is thought to be the physiologic mechanism of action of gastric pacing to effect weight loss.

Previously, a single tissue bulking device called ENTERYX™, is marketed for treating gastro esophageal reflex disease, GERD. With the ENTERYX device, the procedure, injects a tissue bulking agent into the gastric muscularis at the gastro esophageal junction (GEJ). This injection is accomplished via the single operating channel gastric endoscope with a penetrating hollow needle on the distal end. The actual injection technique is to stabilize the GEJ by maximally inflating the stomach with air, and abruptly penetrate the gastric mucosa and muscularis with the distal needle. Two complications have occurred because of this technique: an inadequate depth of penetration of the needle; and much more concerning, too deep of a penetration of the needle completely through the full thickness of the gastric wall and into the aorta. This aortic death complication of the procedure even caused a temporary suspension of the ENTERYX procedure by the FDA. The FDA has now recalled that procedure (2005). The complication of “too shallow” of a placement of the tissue bulking agent in the mucosa, instead of the muscularis, caused a leaking of the device into the gastric lumen and caused 6 of the 63 patients to have a second operative procedure to properly place the ENTERYX bulking device into the muscularis. These two complications were thought to be both physician learning and technique oriented.

A. Vagus Nerve Anatomy and Physiology
Gray's Anatomy describes the vagus nerve, the tenth (X) cranial nerve, as the “wandering” nerve. This is also named the pneumogastric nerve. The vagus nerves supply the non-striated muscle (smooth muscle) and exocrine glands of the esophagus and the stomach with general efferent para-sympathetic innervations. In the mediastinum and abdomen, the vagus nerves, right and left, course on the right and left sides of the esophagus, sending off multiple branches to the smooth muscles of the mid and distal esophagus. At the gastro-esphageal junction, the right and left vagus nerves attain a companion course where they are only one or two centimeters apart and course along the lesser curvature of the stomach together. From this position, in the lesser curvature of the stomach, the vagus nerve sends out multiple branches, like the branches of a tree, to innervate and control the Glands and smooth muscles of the stomach. The region of the gastro-esophageal junction and the proximal lesser curvature of the stomach can be described as the control center for the glands and the smooth muscle cells of the stomach. With increased parasympathetic activity there is increased electrical stimulation to the smooth muscles of the stomach, and increased stimulation to the glands of the gastric mucosa. In fact, the surgical treatment for peptic ulcer disease is a vagotomy and antrectomy. The vagotomy is the transsection of the vagus nerve in the lesser curvature of the stomach. This vagotomy causes a drastic decrease in the production of gastric acid from the gastric cells of the gastric mucosa and allows the peptic ulcers to heal and not reform, with an 80-90% success rate. The vagotomy also decreased the muscle tone and activity of the smooth muscles of the stomach and delays the emptying time of the stomach. To compensate for this, the size of the stomach is reduced with the antrectomy, to remove 30-40% of the stomach.
   In development of the vagotomy and antrectomy procedure, it was found that transsection of the vagus nerve above the gastroesophageal junction causes a significant complication of gastroesophageal gastric acid reflux. This gastric acid reflux causes esophageal ulcers because of the decreased muscle tone of the smooth muscles defining the physiological valve between the stomach and the esophagus, the gastroesohageal junction.
   Although vagotomy and antrectomy procedures are significant and effective surgical solutions to peptic ulcer disease, the procedure has fallen out of favor because of the multiple medical therapies to reduce the production of gastric acid and cure peptic ulcer disease. Tagamet (cimetadine) introduced in the 1980's and recently more effective proton pump inhibitors, have relegated the vagotomy and antrectomy procedures to a historical perspective only. It is interesting that several reports in the medical literature document weight loss after vagotomy and antrectomy procedures. In 1978, J. G. Kral reported in Lancet (8059): 307-8 on three women who lost 20-40 pounds after vagotomy only. In 1979, J. P. Mordes et al reported that after vagotomy, thirteen rats reduced weight by 26% compared to controls and again in 1981, J. G. Kral reported the weight reduction of an average of 40 pounds each in twenty one morbidly obese patients after truncal vagotomy.

Anatomy and Vagal Nerve Trunk Identification

The ENTERYX procedure is accomplished at the anatomic landmark, the gastroesophagel junction (GEJ). This junction is even defined by a change in mucosal characteristics easily recognized with the gastric endoscope. In contrast, the vagal nerve trunks are located within the lesser gastric curvature without either a specific anatomic or mucosal landmark. As previously described in our U.S. patent application Ser. No. 11/165,075, the vagal nerve trunks are nerves about the thickness of thick twine, located either subserosably or within the distal gastric muscularis, and course parallel to one another, about five to fifteen millimeters apart in the lesser gastric curvature. The use of an ultrasound probe through the operating channel of a gastric endoscope can locate the vagal nerve trunks, and define the precise injection site midway between the right and left vagal nerve trunks. A post-injection ultrasound would confirm the proper placement of the tissue bulking agent to effect vagal nerve trunk physiology.

### Gastric Wall Grasping/Manipulation

With the present invention, once the precise location for placement of the tissue bulking agent to interface with the vagal nerve trunks is anatomically identified, the gastric wall would be firmly grasped with a specific grasper placed through the operating channel of a gastric endoscope. The grasping instrument would have a standard proximal end and shaft, but the distal end would be a grasping instrument. Once properly positioned, an arrangement of grasper jaws could be locked in place by a proximal handle standard locking mechanism. The gastric wall grasper is necessary because the lesser gastric curvature is supported by the mesentery, not fixed and stable like the gastro esophageal function (by the diaphragm). In the ENTERYX procedure a simple needle penetration of the gastric wall is acceptable because of the diaphragmatic fixation of the injection site. This would not be acceptable in the lesser curvature of the stomach.

### Benefits of the Gastric Wall Raspers

The gastric wall graspers would provide defined benefits that would allow the skilled gastro enterolgist the relatively easy and safe placement of a tissue bulking agent adjacent to the vagal nerve trunks.
Counter tractions for needle penetration of the gastric mucosa and muscularis
Containment of the tissue balking agent after injection
Creation of a submuscularis reservoir

Every medical student is taught to “pick up what you want to sew”. Thumb or tissue forceps are used to stabilize and provide counter traction against a needle placed through tissue for suturing. The gastric wall grasper of the present invention would provide stabilizing tissue for advancement of the tissue onto the injection needle. The FDA suspended the ENTERYX procedure after a woman died when the ENTERYX needle penetrated completely through the gastric wall and lacerated her abdominal aorta. She exsangainated within hours. A contributing factor in this mishap was the needle penetration in non-stabilized tissue.

In the FDA review of the ENTERYX clinical studies, six (6) of sixty three (63) patients had leakage of the ENTERYX material into the lumen of the stomach. These patients required re-operation. The grasper of the present invention would be used to disorient the different tissue layers before needle penetration of the gastric wall and therefore place the tissue bulking device into a closed space once the needle is removed and the tissue layer resume their normal orientations. This method of tissue layer disorientation is used for intramuscular injections. The graspers with the penetration and fixation of all tissue layers, especially the deeply placed muscular layers, by the tissue being advanced onto the injection needle, would afford the needed tissue layer disorientations for secure sealing of the tissue bulking device where it is injected.

### BRIEF SUMMARY OF THE PRESENT INVENTION

A Tissue Injection Apparatus: The proximal end of the tissue treatment agent (i.e. bulking agent) delivery apparatus would terminate with a luer lock connection to accept the syringe that typically contains the tissue bulking agent. The luer lock affords the pressurized injection necessary for placement of tissue bulking agents.

The distal end of the tissue bulking delivery injection apparatus would terminate with a defined length of a sequestered small-gauge needle and prevent over-penetration of the needle contained in an elongated housing. The intervening assembly between the distal needle and the proximal luer lock would include a hollow tubular connection for transit of the tissue bulking agent. The proximal end of the housing encloses a thumb-hold actuated plunger having a distal end arranged within the housing, which distal end movably advances and retracts a rotatable, articulable arrangement of tissue grasper arms which are arranged to rotate and open beyond the distal end of the housing to grasp and rotatively return into the housing with tissue distortively pulled therealong into penetrative engagement with a treatment means injection needle secured within the distal end of the housing.

The present invention includes, for example, a gastroscopically placed tissue bulking agent adjacent to the right and left vagus nerve trunks in the lesser curvature of the of the stomach or at the gastroesophageal junction. The intent of the invention is to provide a method and apparatus for the compression of the vagus nerves that control both electrical stimulation of the gastric ghrelin cells and the electrical stimulation of the parastolic muscular tone of the smooth muscles of the stomach. A further embodiment of this invention is to utilize the parastolic activity, although reduced, to cause a dynamic movement of the tissue bulking agent against the vagus nerve trunks. This action of recurrent dynamic vagus nerve compression is mechanical in nature, not like gastric pacing, which is electrical, electromechanical or both. The mechanical recurrent vagus nerve compression utilizes the antral pump action to force the peri-vagal bulking agent against the vagus nerves. The peri-vagal bulking agent can be any of the known tissue bulking agents, for example: collagen, Teflon, macroplastique, micro-carbon particles, silicon microspheres or Ethylene/vinyl alcohol co-polymer (Onyx). The method of placement of the tissue bulking agent in a peri-vagal position would be best described by the ENTERYX procedure by Boston Scientific—which is patented and FDA approved for treatment of GERD (gastroesophageal reflux disease). Further information, incorporated herein by reference, can be obtained on www.reflux1.com.

A separate mechanism of action of the peri-vagal tissue bulking would be to effect and reduce the speed of the antral pump. Previously stated in the anatomy and physiology section hereinabove, the vagus nerve is responsible for both glandular and muscular activity of the stomach. By reducing the smooth muscle tone and by reducing the cycle of the antral pump, food will exit the stomach more slowly adding to the sensation of prolonged stomach fullness and satiety. In fact several reports in literature attribute weight loss to exactly this mechanism with injections of the neuromusculature blocking agent Botox, directly into the smooth muscles of the stomach wall. Gui et al reported in January 2000, Ailment Pharmacology Theory, “Botulisum Toxin Injected in the Gastric Wall reduces body weight and food intake in rats.”

An additional embodiment of this invention would be, for example, the use of a piezoelectric material used as the tissue bulking agent. In addition to the mechanical forces of the bulking agent effecting the vagus nerves, the mechanical forces exerted on the bulking agent by the stomach would produce a small electrical current to effect on the vagus nerves, similar to that produced in a gastric pacing procedure. The electrical stimulation of the vagus nerve is described best in gastric pacing procedures where electrodes are placed in the lesser curvature of the stomach and apply currents to the smooth musculature and vagus nerves. For purposes of the present invention, the term “piezoelectric composition” as used herein, is intended to encompass for example, natural and synthetic materials which are capable of generating electrical charges on their surface when subjected to mechanical strain. Thus, for purposes of the present invention, any material which generates an electrical charge in response to a mechanical strain is to be considered a piezoelectric composition. When used in conjunction with the present invention, such piezoelectric compositions must be biodegradable.

Suitable piezoelectric compositions include, for example, polypeptide polymers, electric polymers and ferroelectric polymers. Other suitable piezoelectric compositions for use with the implantable members of the present invention include, for example, biodegradable polyepsilon amino caprolactone, polyhydroxybutyrate, polyvinylidene fluoride, polyvinyl fluoride, vinylidene fluoridetrifluoroethylene copolymer, vinylidene cyanide-vinyl acetate copolymer, polyvinyl chloride, polylactic acid, collagen, nylon 11, polygamma benzylglutamate, polygamma methylglutamate, copolymers of trifluoroethylene, copolymers and derivatives thereof.

The invention thus comprises an apparatus for the retentive placement of a treatment agent in a body tissue, comprising: an elongated housing; a treatment-agent-supplying hollow injection needle supported in a distal end of the elongated housing; an arrangement of articulable tissue grasping arms movable within said distal end of said housing to grasp, pull and distortively twist the tissue into the distal end of said housing; and a movement connector for rotatively and longitudinally moving the grasping arms out of and back into the distal end of the housing. The injection needle may be curved. The treatment agent is preferably a bulking agent for introduction adjacent to the vagal nerve of a body. The movement connector may have a cam thereon to follow a spiraled track in the distal end of the elongated housing to effect rotation of the connector and the grasping arms about a longitudinal axis of the housing. The spiraled track in the distal end of the housing preferably has a curve which generally corresponds to the curve of the injection needle. The grasping arms are in a sliding relationship with an inner edge of the distal end of the housing. The rotational and longitudinal movement of the connector is effected by longitudinal movement of a connected, operable thumbhold and plunger on a proximal end of the housing. The treatment agent is introduced into the housing through a luer lock in the housing, the luer lock being in fluid communication with the hollow injection needle.

The invention also comprises a method of placement of bulking materials adjacent to the vagal nerve in a body, comprising one or more of the steps of: introducing an elongated tissue grasping apparatus through a scope into a body; rotating in a first direction and longitudinally advancing a grasping arm arrangement through the apparatus and toward the vagal nerve; splaying open the grasping arm arrangement as the grasping arm arrangement moves out of the distal end of the housing of the apparatus; grasping the tissue adjacent the vagal nerve; pulling and rotating in a reverse or second direction, the grasping arm arrangement back within the apparatus so as to distort the tissue; injecting the tissue with the bulking materials fed to a stationary needle after and as the distorted tissue is being rotatably withdrawn into the apparatus. The method includes rotatively advancing in the first direction the distorted injected tissue distally out of the apparatus.

The invention also comprises a method of introducing treatment materials into body tissue while minimizing leakage of treatment materials therefrom, comprising: introducing a treatment apparatus into the body through an endoscope; rotatively and longitudinally advancing a tissue grasping arm arrangement out of the apparatus housing and against a tissue to be treated; grasping and rotatively and longitudinally returning the tissue back into the apparatus to as to distort said tissue; injecting the distorted tissue through a stationary (or a rotatively and longitudinally advancable needle in a further preferred embodiment) needle arranged with a distal end of said apparatus; and controllably rotatively un-distorting and returning the treated tissue to its place outside of the apparatus. The needle may be curved. The tissue may comprise vagal nerve tissue. The distortion of the tissue and the injection of the tissue may occur preferably in a stepped procedure or it may occur simultaneously. The treatment material may comprise a bulking agent. The treatment material may comprise a tissue distorting piezoelectric material. The tissue pre-injection and/or during injection is preferably distorted along a spiral curved path which preferably corresponds to the general curvature of the injection needle.

The invention also includes a method of providing a treatment agent to a body tissue comprising one or more of the steps of: rotatably advancing a grasper arrangement from a treatment apparatus; grasping the tissue to be treated; rotatably pulling and simultaneously injecting the grasped, now initially pre-distorted tissue in the apparatus; rotatably dischargably distally advancing and un-graspingly returning the treated distorted tissue into its natural undistorted configuration beyond the housing of the apparatus. The injecting-the-grasped-tissue may occur with a hollow, preferably curved treatment-agent needle. The rotation path and the hollow curved needle are of similar curvilinear characteristics.

### BRIEF DESCRIPTION OF THE DRAWINGS

The objects and advantages of the present invention will become more apparent when viewed in conjunction with the following drawings, in which:

FIG. 1 is a side elevational view, partly in section, of a tissue distorting and injection apparatus constructed according to the principles of the present invention, showing that apparatus in an initial configuration;

FIG. 2 is a view similar to FIG. 1, showing that apparatus with grasping arms grasping tissue from a distal end thereof;

FIG. 3 is a view similar to FIG. 2, showing that apparatus grasping and capturing a portion of tissue by an articulated arrangement of grasping arms extending therefrom;

FIG. 4 is a view similar to FIG. 3, showing tissue withdrawn and captured within the distal end of the apparatus, relative to a stationary needle therewithin;

FIG. 5 is a view similar to FIG. 4, showing the injection of a treatment agent into that captured tissue within the distalmost end of the apparatus;

FIG. 6 is a view similar to FIG. 5 showing the release of that treated tissue from the distalmost end of the capturing apparatus;

FIG. 7 is a view similar to FIG. 6 showing a pair of grasper arms fully releasing the tissue and showing the tissue treatment agent left within that tissue from which it had been held; and

FIG. 8 is a view similar to FIG. 1 showing the apparatus with its grasper arms withdrawn within its distalmost end permitting further procedures at the tissue treatment site.

### DETAILED DESCRIPTION OF THE PREFERRED EMBODIMENTS

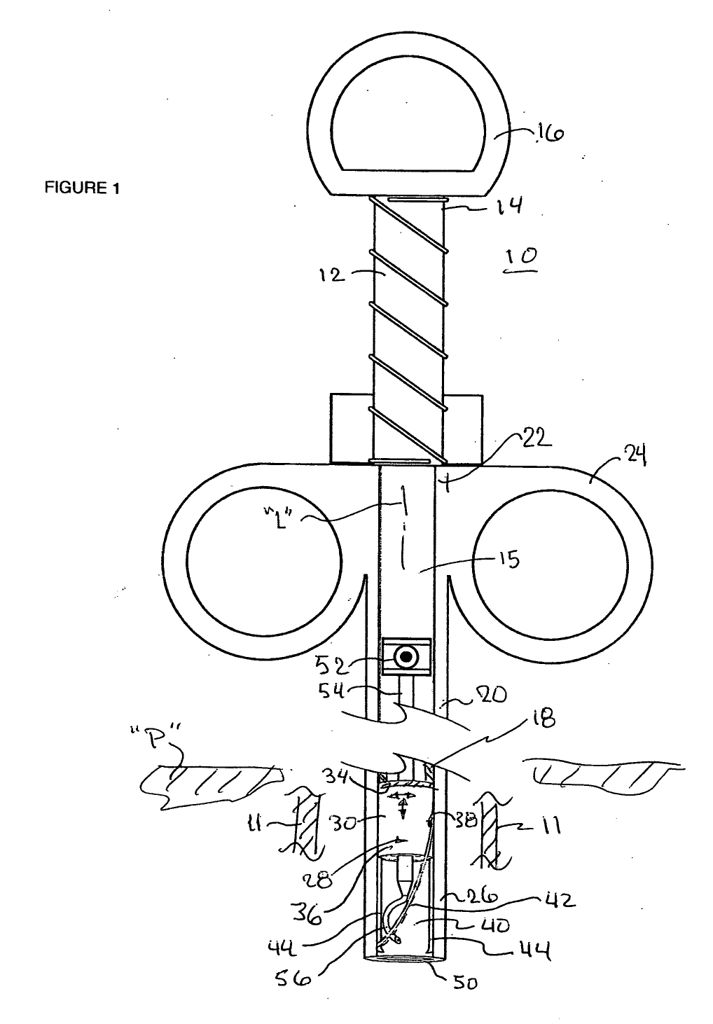

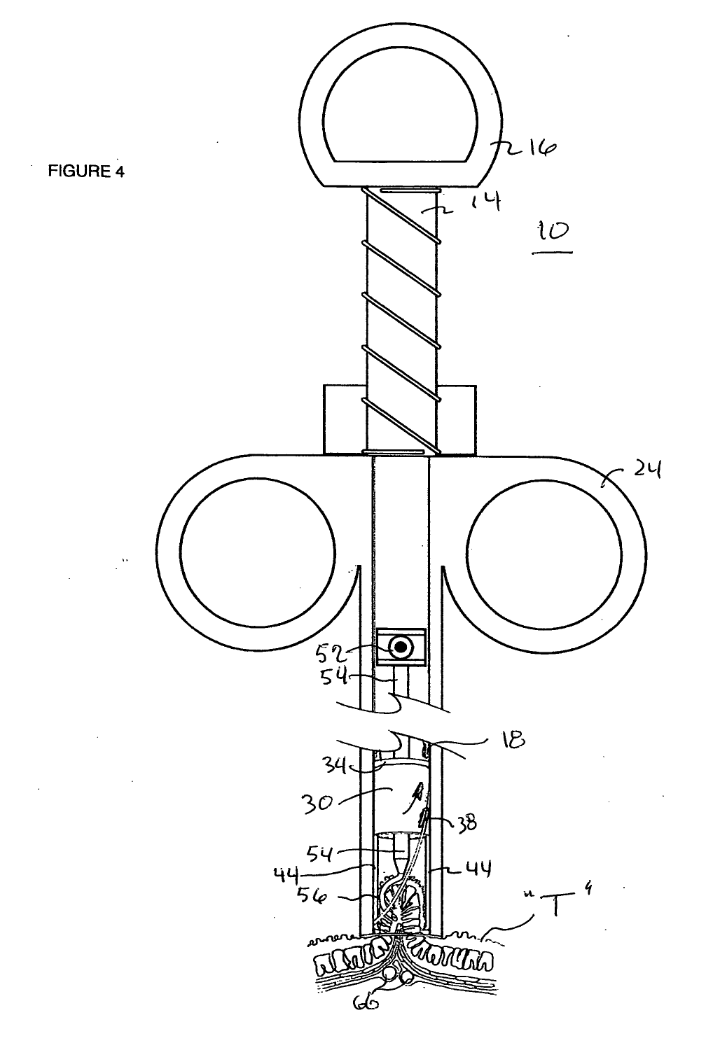

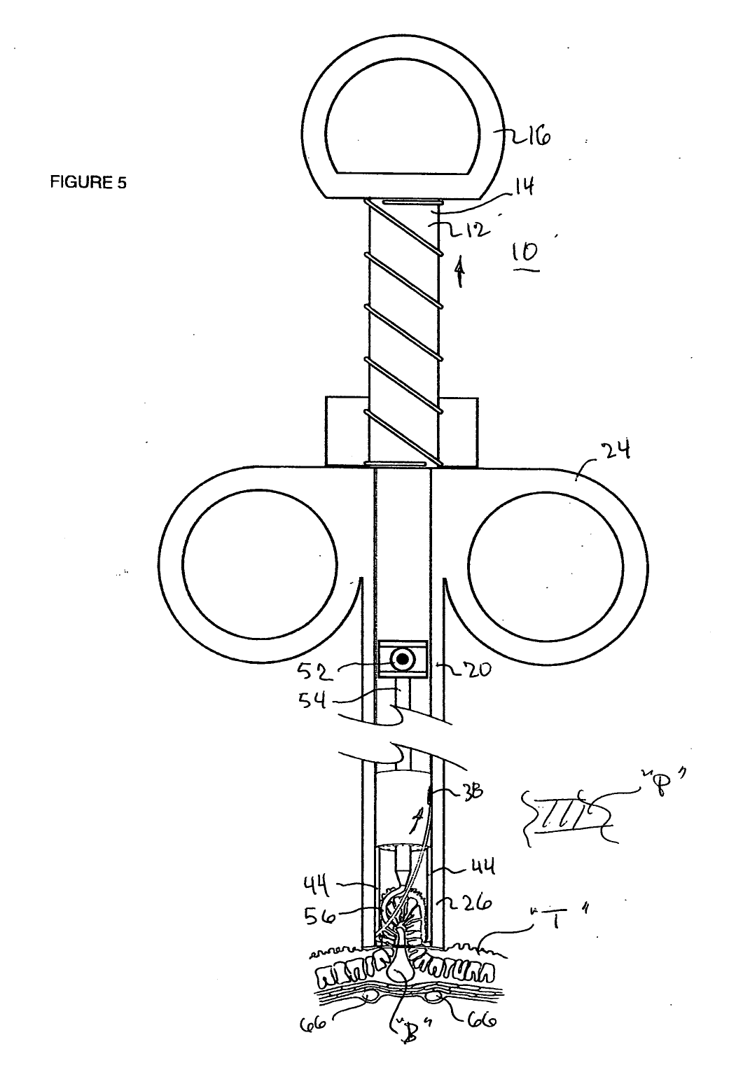

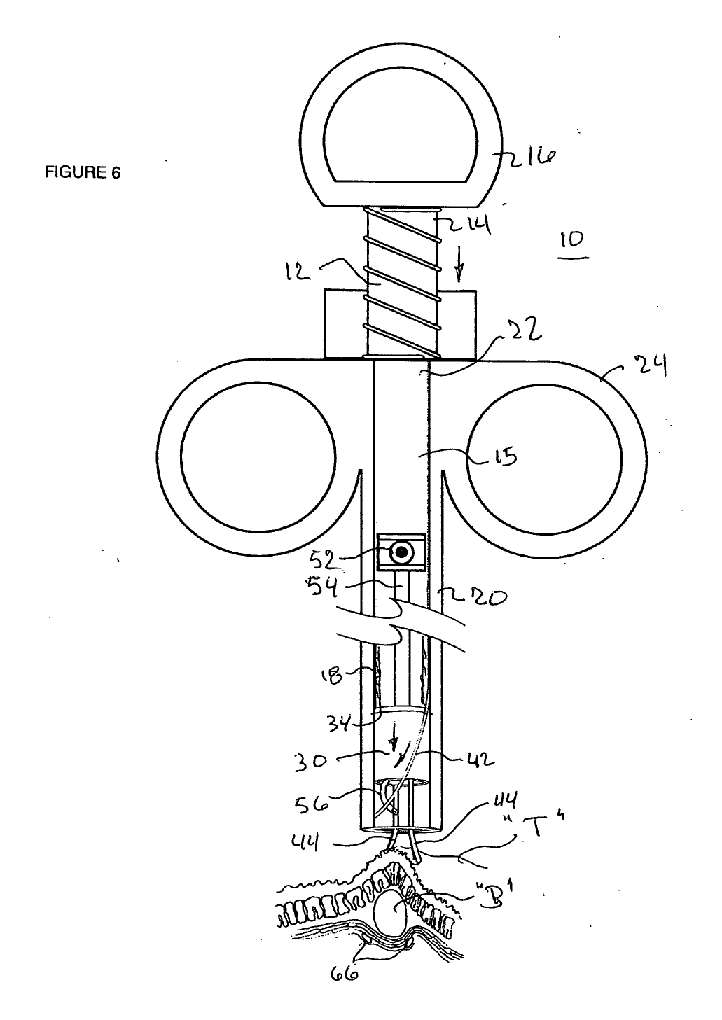

Referring now to the drawings in detail, and particularly to FIGS. 1-8, there is shown the present invention which comprises a tissue distorting and injection apparatus 10, insertable into a patient “P” through an endoscope 11, the apparatus 10 comprising an elongated axially displaceable plunger 12 having a proximal end 14 with a thumb hold 16 thereon arranged in communication with a barrel shaped housing 15 having a longitudinal axis “L”. The plunger 12 has a distal end 18 which is slidably disposed within an elongated outer sleeve or housing 20 of the apparatus 10. The outer sleeve 20 has a proximalmost end 22 with a finger hold 24 thereon. The outer housing or sleeve 20 has a distalmost end 26 which encloses a movable tissue grasping and injection arrangement 28.

The tissue grasping and injection arrangement 28 comprises a displaceable guide ring 30 which is rotationally and longitudinally movably arranged on the distal end 18 of the sleeve 20. The distal end 18 of the plunger 12 is longitudinally displaceable within the outer housing or sleeve 20 of the tissue distorting and injection apparatus 10.

The guide ring 30 is a generally annularly shaped housing, and is both longitudinally and angularly displaceable simultaneously by virtue of a rotatable connector 34 arranged therebetween at the distal end 18 of the plunger 12.

The guide ring 30 has an annular outer surface 36 with at least one cam projection 38 extending radially outwardly thereon. The outer sleeve 20 has an inner surface 40 with a spiral cam track groove or rifling 42 spirally disposed therein. Longitudinal movement of the guide ring 30 via the surface 40 effected by the displacement of the plunger 12 effects spiral movement of the cam 38 within the cam track or groove 42 in spiraling displacement around its longitudinal axis “L”. Thus, the guide ring 30, in its reciprocal movement also rotates about its longitudinal axis “L” by preferably about at least 90 degrees.

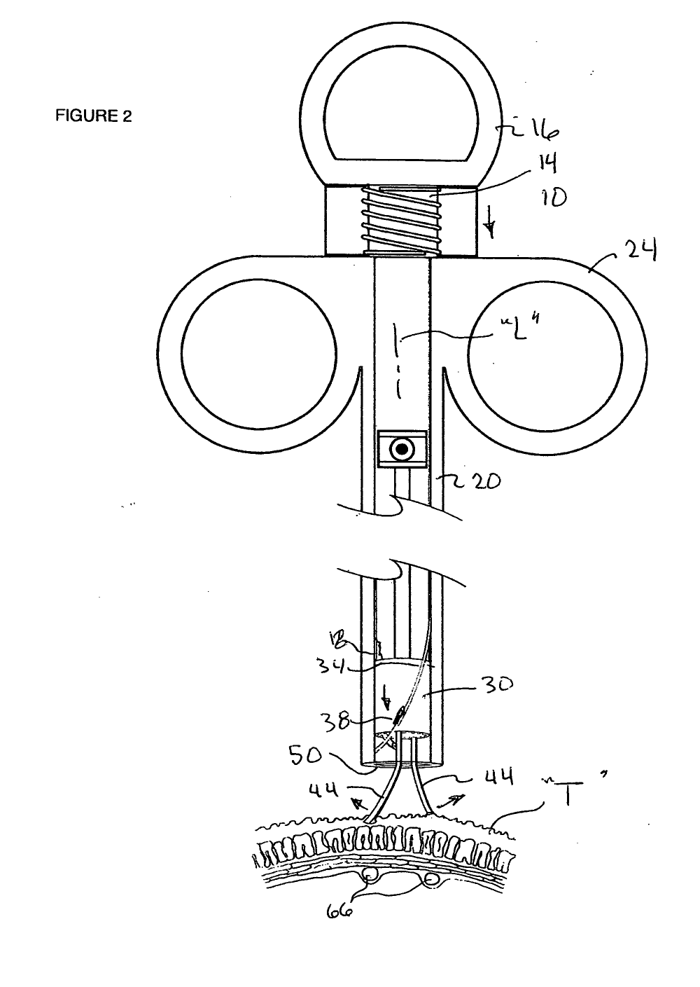

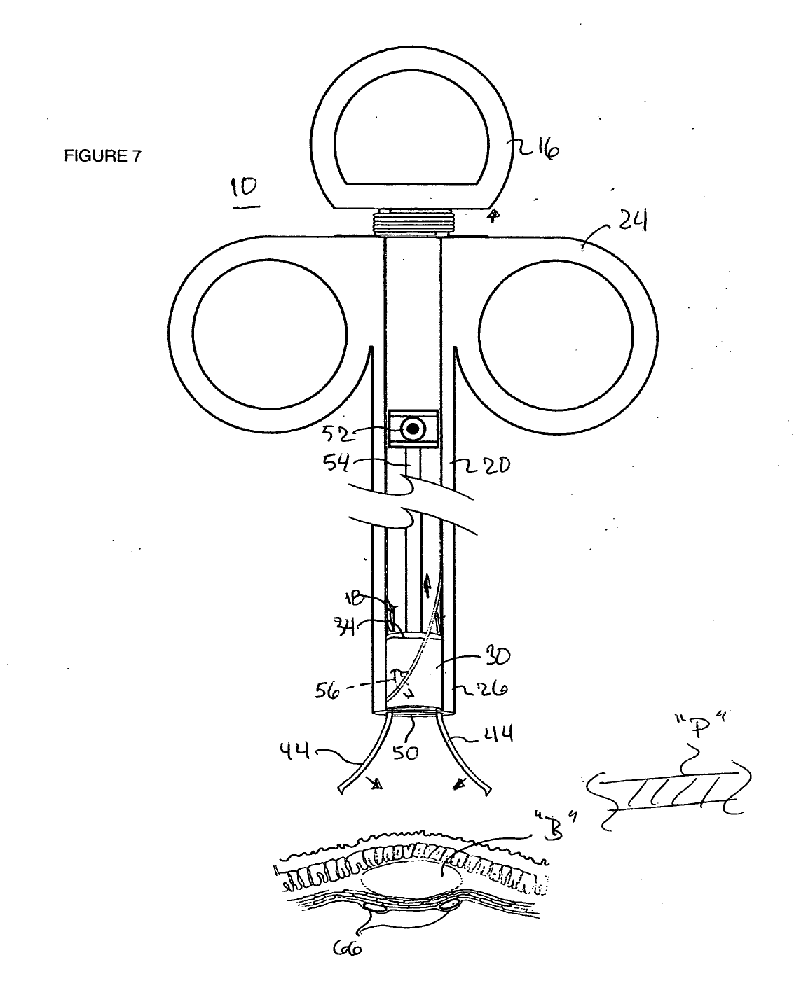

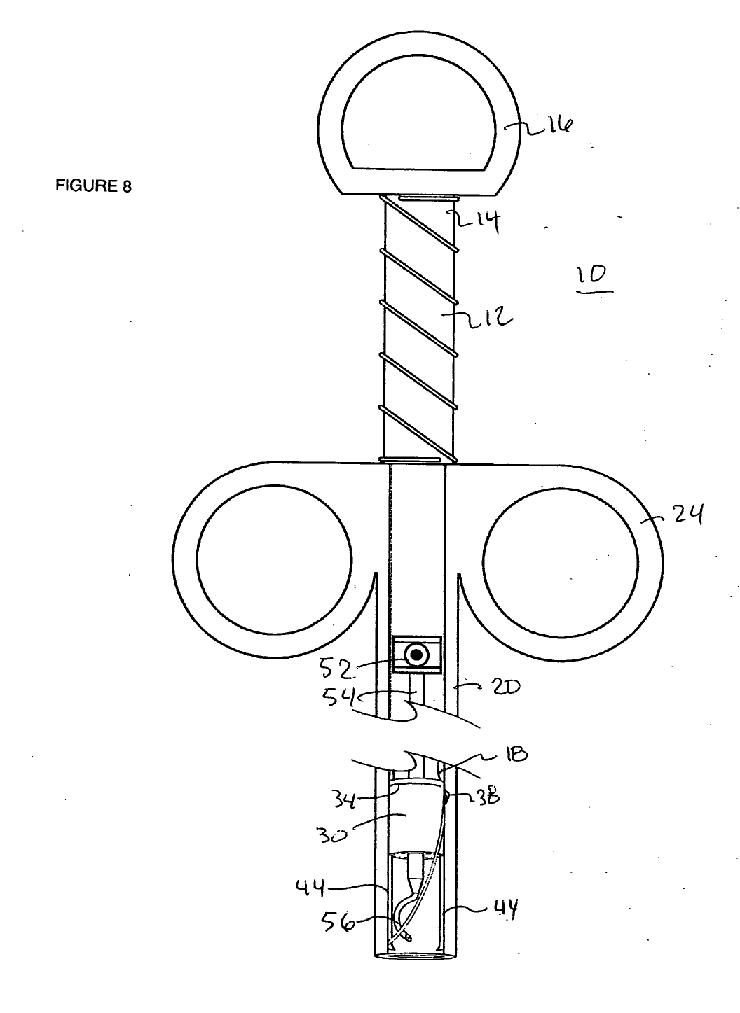

An arrangement of radially outwardly biased grasper arms 44 are secured to the distalmost end of the guide ring 30, as best represented in FIG. 2. Longitudinal displacement of the guide ring 30, as represented between FIGS. 1 and 2, and between FIGS. 7 and 8, effects longitudinal displacement of the grasper arms 44. As those grasper arms 44 are displaced longitudinally, they are permitted to spread outwardly radially with respect to sliding control on the distalmost lip 50 on the inner edge of the outer sleeve 20, as best represented in FIG. 7. Advancement of the guide ring 30 towards the distal end 26 of the outer sleeve 20, permits those grasper arms 44 to bias themselves outwardly, as may be seen in its difference in distal position represented between FIGS. 2 and 7. Displacement of the guide ring 30 proximally, by action of the thumb hold 16 being displaced proximally, effects a biasing inwardly of those grasper arms 44 as they are pulled within the distalmost end 26 of the outer sleeve 20, as represented between FIGS. 7 and 8.

A luer lock port 52 is arranged through the outer sleeve 20 and is stationary with respect thereto. The luer lock 52 has a innermost channel 54 having a distalmost end which is in supply communication an ejection needle 56. The distalmost end of the injection needle 56 is sequestered preferably stationarily within the distalmost end 26 of the outer sleeve or housing 20, as represented in FIGS. 1, 2, 3, 4, 5, 6 and 8.

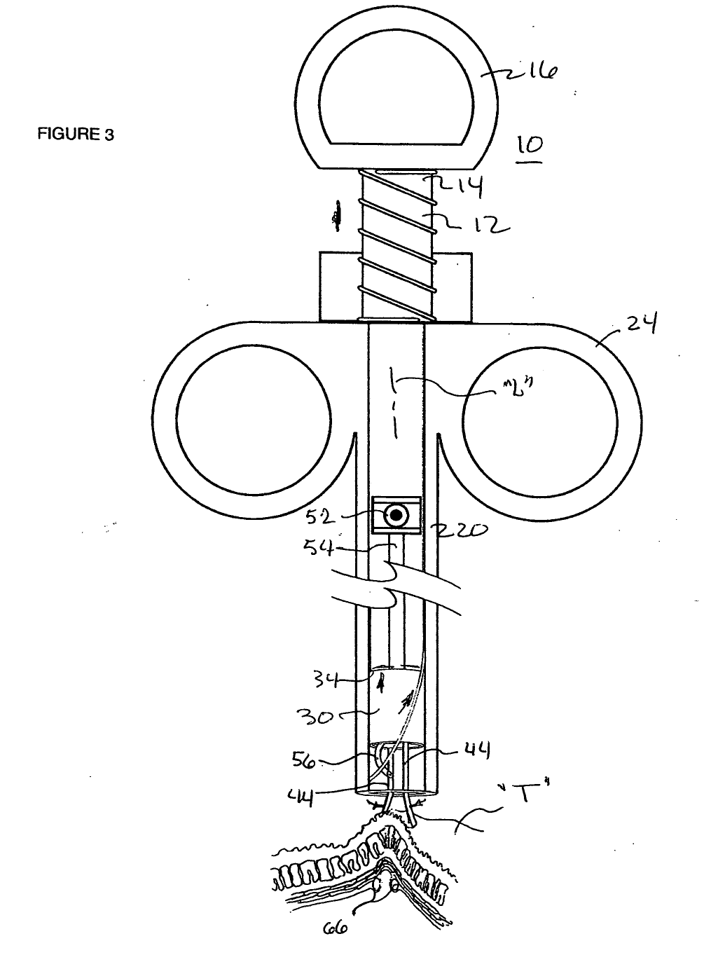

In one preferred embodiment, the injection needle 56 is curved, and rigidly disposed so as to effect an angular injection site in any tissue “T” it engages, as represented in FIGS. 3, 4 and 5 wherein the sequestered needle 56 is held steady and the tissue “T” (i.e. gastric mucosa and muscularis 58) is rotated and hence distorted by the grasping arms 44 pre and during impaling by that curvilinear needle 56. The needle 56 and the spiral track groove 42 are of similar “hand”, having generally corresponding curves to facilitate piercing of and withdrawal of the tissue “T” from the needle 56 upon twisting motion of the tissue “T” with respect to the needle 56 within the distal end of the housing 15 (outer sleeve 20).

The outwardly biased grasper arms 44, typically made of spring metal, expand radially outwardly as they are displaced distally, restricted only by the inner periphery 50 of the distal end 26 of the outer sleeve 20, in physician-operated thumb hold movement, advanced to engage tissue “T” such as for example a capture of the gastric mucosa and muscularis, as represented in FIG. 2. Further, proximal movement of the plunger 12 will effect proximal movement of the guide ring 30 as well as effect angular rotative rotation of the guide ring 30 about its longitudinal axis “L”, so as to withdraw the tissue “T” from the (curved) needle 56 as the grasping arms are withdrawn into the distal end 26 of the outer sleeve 20.

In the injection process, the grasped and captured gastric mucosa (“T”) is itself compressed and rotatably and distortively advanced into the distal end of the sequestered injection needle 56. That rotational movement of the guard ring 30 and hence the rotational movement of the mucosa effects the piercing of that mucosa and muscularis by the curvulinear injection needle 56 which corresponds to the counter rotation of that guide ring 30. The distortion and forcing of the gastric mucosa and the muscularis onto the curvilinear stationary injection needle 56 permits that disoriented tissue layer “T” to be injected with a treatment agent, for example a bulking agent “B”, which is injected into, for example a position in the submuscularis which is, for example adjacent to the vagal nerve trunks 60, represented in FIGS. 2 and 7, and to the gastric serosa. Such a bulking agent fed through the lure lock injection port 52 from a source, while the tissue “T” is disoriented, permits that same tissue “T”, when it returns to its oriented state as to be withdrawn from the distal end 26 of the outer sleeve 20, thus seals that lumen in the tissue made by the needle 56, which would otherwise might have permitted treatment agent “B” to leak therefrom or therethrough were it not for such distortion of the tissue.

A further injected treatment or bulking agent may comprise, for example, a piezoelectric material (i.e. ribbon or wire) to produce a small electric current effect upon the vagal nerve trunks 66.

Upon delivery of such treatment agents, retraction of the tissue disturbing and injection apparatus 10 through the gastroscope/endoscope 11, as represented in FIG. 8, permits an injection site observation for possible bulking agent leakage and a post procedural ultrasound so as to document the bulking agent and vagal trunk juxtaposition.

We claim:

1. An apparatus for the retentive placement of a treatment agent in a body tissue, comprising:
an elongated housing;
a treatment-agent-supplying hollow injection needle supported in a distal end of said elongated housing;
an arrangement of articulable tissue grasping arms movable within said distal end of said housing to initially grasp, pull and twist and distort said tissue into and subsequently push outwardly and un-distortively release said tissue from said distal end of said housing; and
a movement connector for rotatively and longitudinally moving said grasping arms out of and back into said distal end of said housing.

2. The apparatus as recited in claim 1, wherein said injection needle is curved.

3. The apparatus as recited in claim 1, wherein said treatment agent is a bulking agent for introduction adjacent to the vagal nerve of a body.

4. The apparatus as recited in claim 2, wherein said movement connector has a cam thereon to follow a spiraled track in said distal end of said elongated housing to effect rotation of said connector and said grasping arms about a longitudinal axis of said housing.

5. The apparatus as recited in claim 4, wherein said spiraled track in said distal end of said housing has a curve which generally corresponds to the curve of said injection needle.

6. The apparatus as recited in claim 1, wherein said grasping arms are in a sliding relationship with an inner edge of said distal end of said housing.

7. The apparatus as recited in claim 1, wherein rotational and longitudinal movement of said connector is effected by longitudinally displaced movement of an operable thumbhold and plunger arranged through a proximal end of said housing.

8. The apparatus as recited in claim 1, wherein treatment agent is introduced into said housing through a luer lock in said housing, said luer lock being in fluid communication through a channel, with said hollow injection needle.

9. A method of placement of bulking materials adjacent to the vagal nerve in a body, comprising:
introducing an elongated tissue grasping apparatus through a scope into a body;
rotating and distally advancing a grasping arm arrangement through a housing of said apparatus and toward the vagal nerve;
spreading open said grasping arm arrangement as said grasping arm arrangement is advanced distally outwardly of the distal end of the housing of said apparatus;
grasping the tissue adjacent the vagal nerve;
pulling and rotating said grasping arm arrangement proximally back within said housing of said apparatus so as to distort the grasped tissue;
injecting said distorted tissue with the bulking materials fed to an injection needle as the distorted tissue is being rotatably pulled into said apparatus.

10. The method as recited in claim 9, including:
rotatively distally advancing in the distorted injected tissue out of the housing of said apparatus.

11. A method of introducing treatment materials into body tissue while minimizing leakage of treatment materials therefrom, comprising:
introducing a treatment apparatus into the body through an endoscope;
rotatively and longitudinally distally advancing a tissue grasping arm arrangement out of said apparatus and against a tissue to be treated;
grasping and rotatively and longitudinally pulling the tissue back into said apparatus to as to distort said tissue;
injecting said distorted tissue through a needle arranged within a distal end of said apparatus; and
rotatively un-distorting and returning the treated tissue to its place outside of the apparatus.

12. The method as recited in claim 11, wherein said needle is curved.

13. The method as recited in claim 11, wherein said tissue comprises vagal nerve tissue.

14. The method as recited in claim 11, wherein said distortion of said tissue and injection of said tissue occurs simultaneously.

15. The method as recited in claim 11, wherein said treatment material comprises a bulking agent.

16. The method as recited in claim 11, wherein said treatment material comprises a piezoelectric material.

17. The method as recited in claim 12, wherein said tissue is distorted along a spiral curved path which generally corresponds to the curve of said injection needle.

18. A method of providing a treatment agent to a body tissue comprising:
rotatably advancing a grasper arrangement from a treatment apparatus grasping the tissue to be treated;
rotatably pulling and simultaneously injecting the grasped distorted tissue into the apparatus;
rotatably advancing and ungraspingly returning the treated distorted tissue into its natural undistorted configuration outside of the apparatus.

19. The method as recited in claim 18, wherein said injecting-the-grasped-tissue occurs with a hollow curved treatment-agent needle.

20. The method as recited in claim 19, wherein the tissue rotation path and the hollow curved needle are of similar curvilinear characteristics.
